(19) United States
(12) Patent Application Publication
(10) Pub. No.: US 2003/0225379 A1
(43) Pub. Date: Dec. 4, 2003

(54) Composite stasis valve

(75) Inventors: David Schaffer, Coral Springs, FL (US); Michael Schaffer, Coral Springs, FL (US); Brian Fischer, Minneapolis, MN (US)

Correspondence Address: Schwegman, Lundberg,, Woessner & Kluth, P.A., P.O. Box 2938, Minneapolis, MN, 55402, US

(73) Assignee: MedAmicus, Inc.

(21) Appl. No.: 10/371,190

(22) Filed: Feb. 19, 2003

Related U.S. Application Data
(60) Provisional application No. 60/357,937, filed on Feb. 19, 2002.

(51) Int. Cl. A61M 5/00

(52) U.S. Cl. 604/250

(57) ABSTRACT

A valve for blocking the flow of gas or fluid with or without an instrument in place within the gas/fluid path. The valve includes a seal module having a proximal end and a distal end with a lumen sized to allow the passage of fluids or gases.

### CLAIM OF PRIORITY

This application claims priority under 35 U.S.C. 119(e) from U.S. Provisional Application Serial No. 60/357,937 filed Feb. 19, 2002, which application is incorporated herein by reference.

### TECHNICAL FIELD AND RELATED APPLICATION

This application relates to catheters, in particular to a composite fluid-stasis valve for use with catheters. This application is related to U.S. Pat. No. 5,429,616, which is incorporated by reference herein.

### BACKGROUND

Fluid stasis mechanisms are commonly used to prevent loss of fluids from the insertion site of a catheter or interventional system. The may range, in complexity, from a simple clamp on a length of tubing to complex valve systems with several moving parts. The most common valves consist of a resilient material in compression within a housing or clamping member. An example of such a valve is patentee's prior U.S. Pat. No. 5,429,616 wherein a length of tubular resilient foam has an occludible lumen.

An example of the simplest form is U.S. Pat. No. 6,088,889 where a wire clamp is used to occlude a portion of tubing. The resilient material may have a lumen or slit that allows for the passage of an instrument such as a guide wire or catheter.

Most of the existing devices require the user to manually open or close the valve by adjusting the compression on the resilient material and subsequently opening or closing the lumen. An example of this configuration is commonly referred to as a Touey-Borst valve. The manual operation of existing valves most often requires a twisting motion or a squeezing motion. In many cases the action requires the use of both hands. In addition, the existing valves often do not prevent the immediate backflow from within the fluid path as an instrument is inserted or removed.

The existing devices do not perform a complete seal against leakage in the presence of a wide range of instruments or in the presence of multiple instruments. For instance, a single valve element with no instrument in place is generally not optimized for sealing in the presence of an instrument. Often a combination of seals is employed to address these issues, for instance: U.S. Pat. Nos. 6,083,207 and 6,024,729 employ primary seal portions in combination with “duckbill-valves” or “0” closure valves.

Thus, the problems are complex and involve a balance between closing force, opening force, friction, compression and durability. If a valve is inordinately tight, having a closed lumen, it may not allow the insertion of soft, flexible instrumentation such as a “floppy-tip” guidewire, a delicate laser fiber or a soft-tipped catheter. Some catheters, optical fibers and fluid transmission tubes are very delicate and can be damaged by excessive compression or insertion force.

Accordingly, what is needed is a durable stasis valve that blocks the flow of gas or fluid completely and immediately with or without an instrument in place within the gas/fluid path.

### BRIEF DESCRIPTION OF THE DRAWINGS

FIG. 1 illustrates a perspective view of a stasis valve as constructed in accordance with one embodiment.

FIG. 2 illustrates a perspective view of a stasis valve as constructed in accordance with one embodiment.

FIG. 3 illustrates an enlarged cut away view of a stasis valve as constructed in accordance with one embodiment.

FIG. 4 illustrates an enlarged cut away view of a stasis valve as constructed in accordance with one embodiment.

FIG. 5 illustrates a side cross-sectional view of a seal module as constructed in accordance with one embodiment.

FIG. 6 illustrates an end, cross-sectional view of a seal module chamber as constructed in accordance with one embodiment.

FIG. 7 illustrates a side cross-sectional view of a seal module as constructed in accordance with one embodiment.

FIG. 8 illustrates an end, cross-sectional view of a seal module chamber as constructed in accordance with one embodiment.

FIG. 9A illustrates a schematic diagram of a stasis valve as constructed in accordance with one embodiment.

FIG. 9B illustrates an enlarged schematic diagram of a stasis valve as constructed in accordance with one embodiment.

FIG. 10A illustrates a schematic diagram of a stasis valve as constructed in accordance with one embodiment.

FIG. 10B illustrates an enlarged schematic diagram of a stasis valve as constructed in accordance with one embodiment.

FIG. 11 illustrates a partial cut away view of a detent arrangement inside a housing as constructed in accordance with one embodiment.

FIG. 12 illustrates a side cross-sectional view of seal module with an instrument as constructed in accordance with one embodiment.

FIG. 13 illustrates a side cross-sectional view of seal module as constructed in accordance with one embodiment.

FIG. 14 illustrates a side cross-sectional view of seal module as constructed in accordance with one embodiment.

FIG. 15 illustrates a side cross-sectional view of seal module as constructed in accordance with one embodiment.

FIG. 16 illustrates an end, cross-sectional view of a seal module chamber as constructed in accordance with one embodiment.

FIG. 17 illustrates an end, cross-sectional view of a seal module chamber as constructed in accordance with one embodiment.

FIG. 18 illustrates an end, cross-sectional view of a seal module chamber as constructed in accordance with one embodiment.

FIG. 19 illustrates an end, cross-sectional view of a seal module chamber as constructed in accordance with one embodiment.

FIG. 20 illustrates a perspective view of a seal module as constructed in accordance with one embodiment.

FIG. 21 illustrates a perspective view of a seal module as constructed in accordance with one embodiment.

FIG. 22 illustrates a perspective view of a seal module as constructed in accordance with one embodiment.

FIG. 23 illustrates a cross-sectional view of a seal module as constructed in accordance with one embodiment.

FIG. 24 illustrates a cross-sectional view of a seal module as constructed in accordance with one embodiment.

FIG. 25 illustrates a cross-sectional view of a seal module as constructed in accordance with one embodiment.

FIG. 26 illustrates a cross-sectional view of a seal module as constructed in accordance with one embodiment.

FIG. 27 illustrates a perspective view of a stasis valve and external mechanism assembly in accordance with one embodiment.

FIG. 28 illustrates a sectional view of a seal module in accordance with one embodiment.

FIG. 29 illustrates a sectional view of a seal module in accordance with one embodiment.

FIG. 30 illustrates a perspective view of a seal valve as constructed in accordance with one embodiment.

FIG. 31 illustrates a transparent perspective view of a housing and a seal valve as constructed in accordance with one embodiment.

FIG. 32 illustrates a cross-sectional view of a seal valve as constructed in accordance with one embodiment.

FIG. 33 illustrates a transparent perspective view of a housing and a seal valve as constructed in accordance with one embodiment.

FIG. 34 illustrates a cross-sectional view of a seal valve as constructed in accordance with one embodiment.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the invention. Therefore, the following detailed description is not to be taken in a limiting sense, and the scope of the invention is defined by the appended claims and their equivalents.

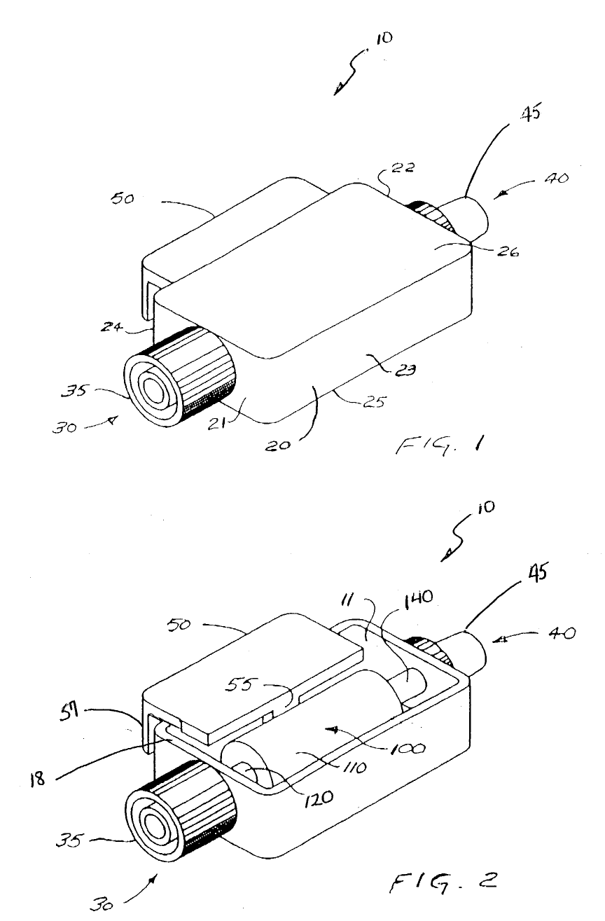

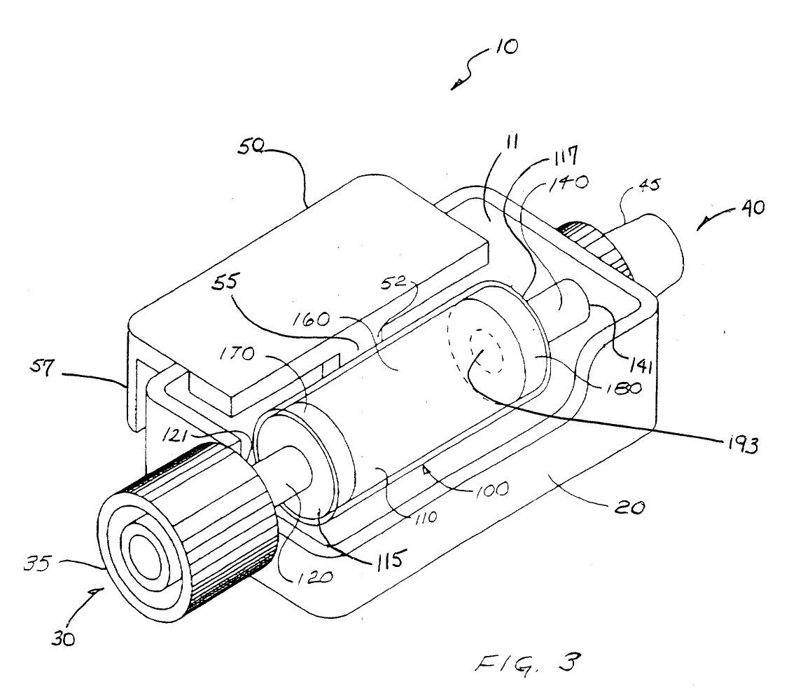

With reference to the drawings, FIGS. 1-4 illustrate a composite fluid stasis valve 10 with a housing 20, a proximal end 30, and a distal end 40. In one embodiment, the housing 20 comprises a hollow rectangular structure having a first end-wall 21, a second end-wall 22, a first side-wall 23, a second side-wall 24, a bottom or floor 25, and a top or lid 26. A hollow interior wall 11 of the housing 20 is sized and configured to hold and control a composite seal module 100, a portion of an actuator 50, and an actuating member 55.

The first end-wall 21 of the housing 20 is fitted with a connecting member 35 sized and configured to attach in fluid communication to a fluid delivery supply or a body passage such as a blood vessel. The connecting member 35 is a common male thread “Luer” type fitting or a common “slit-fit” tube connector or the like. The second end-wall 22 of the housing 20 is sized and configured to receive an inserted instrument, catheter or guide wire through a receiving member 45.

The actuator 50, in one option, includes an actuator flange 57 exterior to the interior wall 11 about the second side wall 24 of the housing 20. In one option, a second stationary member 65 is positioned in the interior wall 11 of the first side wall 23 of the housing 20 distal to the actuating member 55. In one example, the second stationary member 65 is part of the interior wall 11 of the first side wall 23 of the housing 20 or in another example, the second stationary member 65 is inserted into the bottom 25 of the housing 20 as a separate piece.

The stasis valve 10 includes the seal module 100 enclosed in the housing 20 such that the seal module 100 is proximally connected to the connecting member 35 and distally connected to the receiving member 45. The receiving member 45 is, in one option, configured to connect to a fluid or gas delivery system or device such as a syringe, intravenous system or the like. The top edge 18 of the second side wall 24 of the housing 20 forms a guide support for moving the actuating member 55 which, in one option, includes an extension 52. The top 26 of the housing 20 provides an opposing support member for the moving actuator 50. As the actuator flange 57 is depressed, the actuator 50 moves across along the top edge 18 of the second side wall 24 toward the interior wall 11 of the first side wall 23 of the housing 20. The actuating member 55 of the actuator 50 depresses and at least partially collapses, a portion of the seal module 100. The collapsed portion of the seal module 100 forms a seal 200 preventing fluid and/or gasses communication between the connecting member 35 and the receiving member 45.

The actuator 50 is adapted to slide from a first position to a second position. In the first position the actuator 50 is, in one option, disposed and held against a portion of the seal module 100 which depresses and at least partially collapses, for example, the central portion 110 of the containment structure 160 by a compressive force 67 from a resilient member (e.g., by a spring 210). In one position, the containment structure 160 has a normally closed position (i.e., the lumen remains sealed until a user depresses the actuator 50). In the second position, the actuator 50 is disposed away from a portion of the seal module 100 by a compressive force 67 (e.g. by depressing the actuator flange 57) thus allowing, for example, the central portion 110 of the containment structure 160 to retract to an unsealed configuration.

A seal module 100, in one option, extends between the first end-wall 21 of the housing 20 and the second end-wall 22 of the housing 20 and is in fluid communication with the connecting member 35 and the receiving member 45. The seal module 100 comprises an elongate tubular structure 101 having a central portion 110, a first end portion 120, and a second end portion 140. The central portion 110 is sized and configured to hold a plurality of sealing members including a first seal member 170, a second seal member 180, and a third central seal member 165. It should be noted that one or more of the first seal member 170, the second seal member 180, and the third central seal member 165 can be formed of the various materials, and/or having the various properties, discussed throughout this application. In one option, the seal module 100 includes the first seal member 170 fixed at a proximal end 115 of the seal module 100, a second seal member 180 fixed at a distal end 117 of the seal module 100, and a third central seal member 165 extending between the first and the second seal members 170, 180. The plurality of seal members 165, 170 and 180 have an internal diameter sized to allow the passage of fluids or gases therethrough.

In one embodiment, the first end portion 120 includes a distal end 121 that axially communicates with the central portion 110 of the containment structure 160 within the hollow interior wall 11 of the housing 20 and axially communicates with the connecting member 35 exterior to the housing 20. The first end portion 120 includes, in one option, a first diameter substantially smaller than the diameter of the central portion 110. The second end portion 140, in one option, includes a distal end 141 that axially communicates with the central portion 110 of the containment structure 160 within the hollow interior wall 11 of the housing 20 and axially communicates with the receiving member 45 exterior to the housing 20. The second end portion 140 includes a second diameter that is substantially smaller than the diameter of the central portion 110. An amount of compressive force 67 is applied to the actuator flange 57 of the actuator 50 by the user causing the actuator 50 to slide across along the top edge 18 of the second side wall 24. As the actuator 50 slides across along the top edge 18 of the second side wall 24, the actuating member 55 is forced against the outer wall 27 of the seal module 100.

In another embodiment, the actuator 50 is adapted to slide from a first position to a second position. In the first position the actuator 50 is disposed and held against a portion of the seal module 100 which depresses and at least partially collapses, for example, the central portion 110 of the containment structure 160 by a compressive force 67 (e.g. by a spring 210) creating a seal 200 preventing gas and/or fluid from passing therethrough. In the second position, the actuator 50 is disposed away from a portion of the seal module 100 by a compressive force 67 (e.g. by depressing the actuator flange 57) thus allowing, for example, the central portion 110 of the containment structure 160 to retract to an uncollapsed configuration. When there is no longer a compressive force 67 (e.g. by releasing the actuator flange 57), the actuator 50 reengages the portion of the seal module 100 and at least partially collapses, for example, the central portion 110 of the containment structure 160.

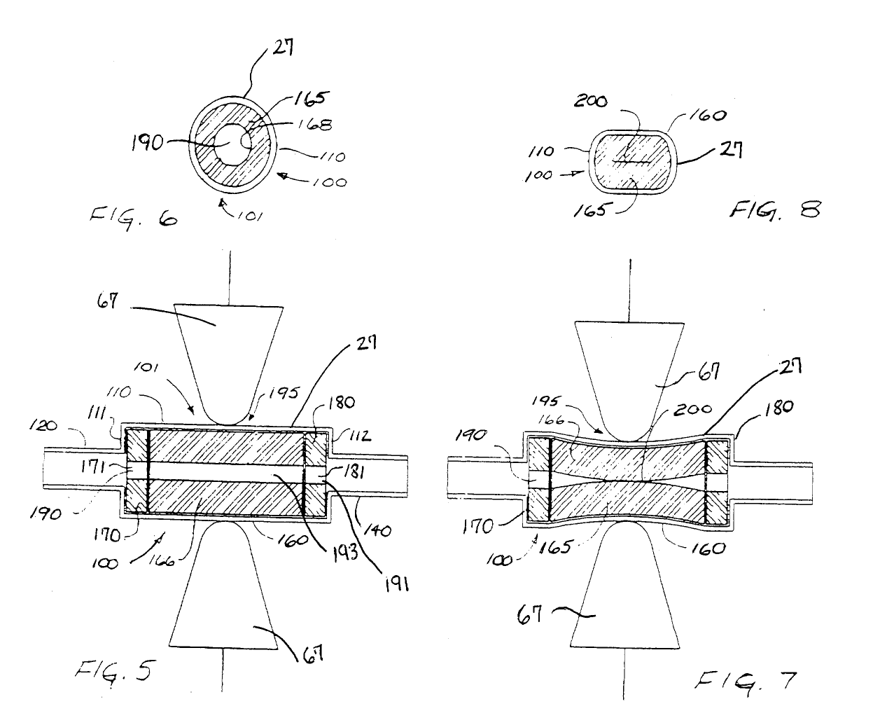

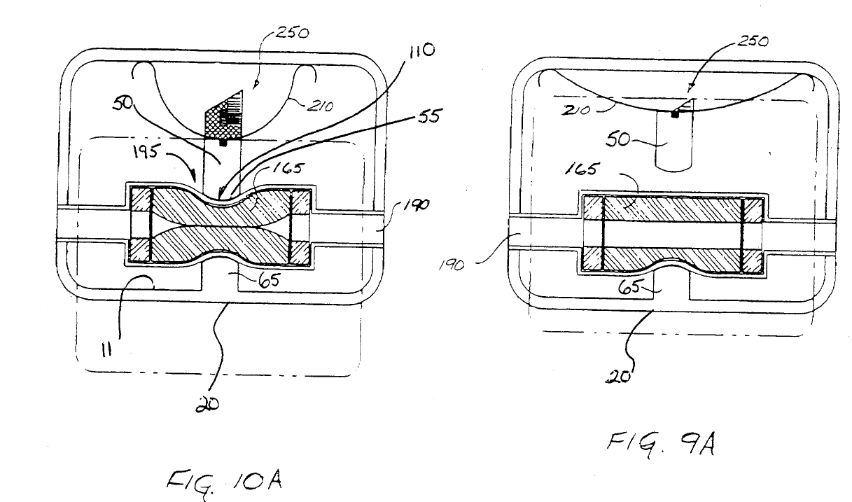

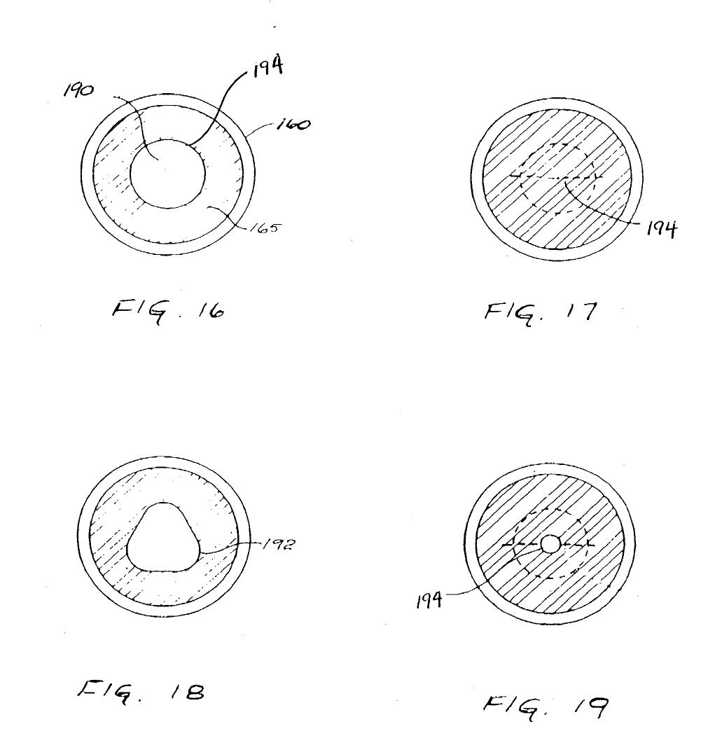

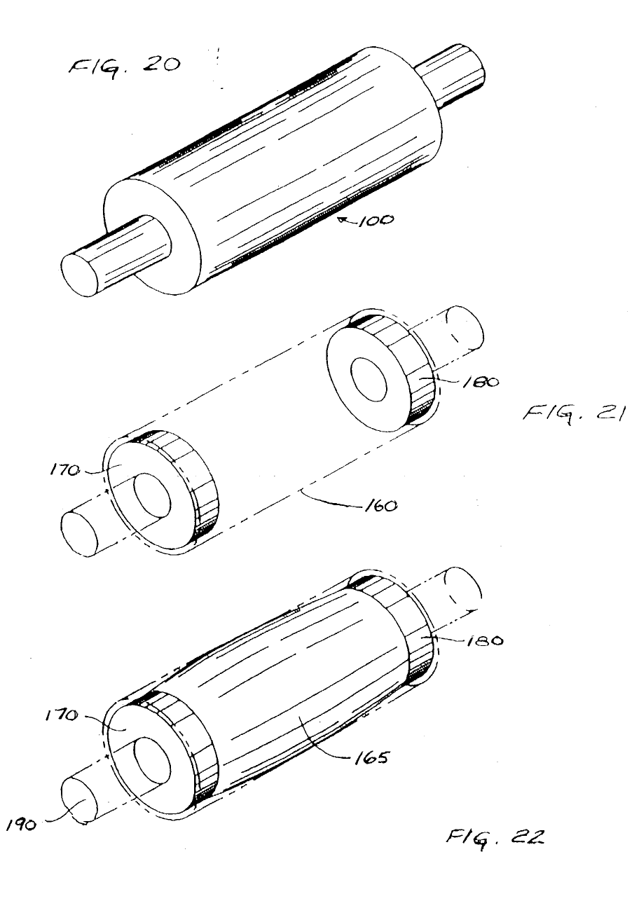

With reference to FIGS. 5-22, the seal module 100, in one embodiment, includes a flexible, elongate tubular structure 101 having an outer wall 27 which includes a material 166 that is highly elastic, deformable, compliant and yet virtually non-compressible. The outer wall 27 is formed so as to have a large diameter in the central portion 110 and a reduced diameter at the first end portion 120 and the second end portion 140 of the seal module 100. A first abutment 111 and a second abutment 112 are formed by the diameter reduction of the elongate tubular structure 101. The first abutment 111 forms a stop or seat for a first seal member 170 and the second abutment 112 forms a stop or seat for a second seal member 180. A third central seal member 165 is placed between the first seal member 170 and the second seal member 180 and in fluid communication therewith. The third central seal member 165 includes a highly deformable, non-compressible material 166 (e.g., plastic). The third central seal member 165 is sized and configured to maintain an open lumen 193 when no compressive force 67 is applied.

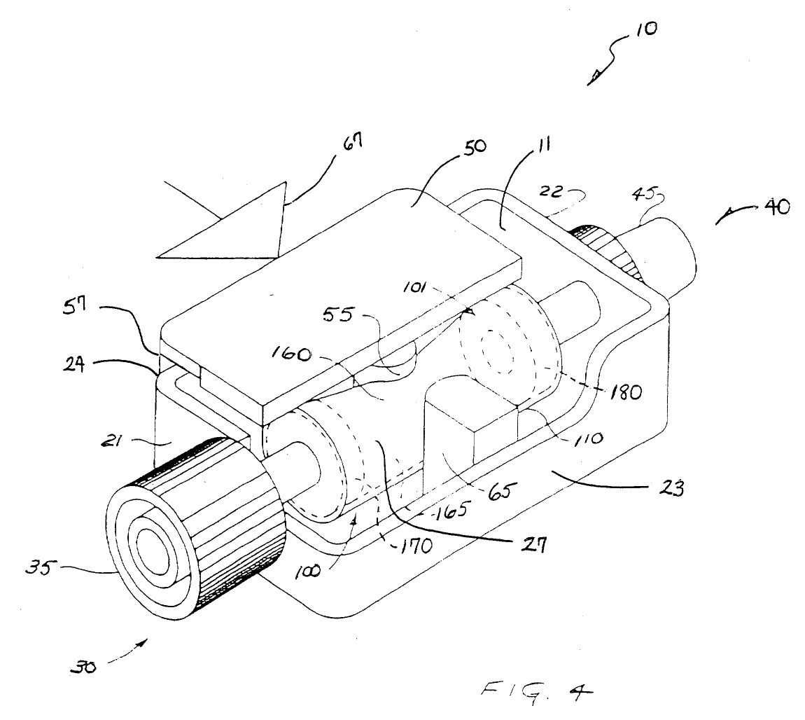

When the actuating member 55 is, in one option, forcibly pushed against the central portion of the seal module 100, the compressive force 67 of the actuating member 55 against the outer wall 27 of the containment structure 160 inwardly depresses or collapses the third central seal member 165 of the containment structure 160 as the actuator 50 progresses toward the first side wall 23 of the housing 20. The third central seal member 165 is, in one option, depressed to the point where the containment structure 160 of the seal module 100 slows or stops the flow of fluid (e.g., blood) from communicating between the connecting member 35 and the receiving member 45 of the stasis valve 10. This creates a seal 200 between the orifices 171, 181 of the lumen 191, 190. The stationary member 65 (see FIG. 4) further assists the depression of the outer wall 27 of the containment structure 160 on an opposing side as the actuating member 55 progresses toward the first side wall 23 of the housing 20.

In one embodiment, the first seal member 170 has an orifice 171 of a selected diameter 194 that corresponds, for example, to a range of instruments used within the seal module 100. The second seal member 180 includes the orifice 181 that corresponds to a range of inserted instruments. The first seal member 170 provides a fluid/gas tight seal around and upon an instrument within a selected range of diameters 194, such as a catheter, guidewire, needle or fiber, inserted within the orifice 171 of the first seal member 170. The second seal member 180 is sized and configured to provide containment for the third central seal member 165. The orifice 181 of the second seal member 180 is, in one option, substantially the same as the orifice 171 of the first seal member 170 and provides a backup or secondary seal in the event that the first seal member 170 becomes damaged.

The first and second seal members 170 and 180 include elastomeric materials, such as rubber or silicone, and are essentially septums sized and configured to seal against gas or fluid pressure around an instrument. The first and second septum seal members 170 and 180, allow smooth and accurate movement of instruments since there is no additional compressive force or load required to complete the seal. In one option, a relatively high durometer material is used as the septum material for the first and second seal members 170 and 180 because it provides a low frictional coefficient against most inserted instruments while providing a competent seal. In another embodiment, one or more of the first, second, and third seal members 170, 180, and 165 includes self-lubricating, lubricious or coated septum materials. Such materials include specialty silicones, natural latex, various synthetic rubbers or elastomeric compounds of polyurethane, vinyl or the like. The low friction nature of the first and second seal members 170, 180 is in contrast to the highly deformable and compliant nature of the third central seal member 165.

The third central seal member 165 includes an elongated tubular structure 101 sized and configured to fit into the tubular containment structure 160 between the first seal member 170 and the second seal member 180. The lumen 193 of the third central seal member 165 is, in one option, slightly larger than the orifice 171 of the second seal member 180 so that an inserted instrument 260 need not contact the luminal surface.

In one embodiment, the third central seal member 165 includes material 166 that is highly elastic, deformable, compliant and yet virtually non-compressible. Materials 166 include modified vinyl, silicone, polyurethane or a combination thereof. The basic materials are, in one option, modified by compounding them with waxes and/or oils or un-cross-linked modifiers. Such materials are commonly available as “C-Flex” or “Kraton” in the range of 5 to 15 (shore A), as examples. The shore hardness of the material 166 is, in another option, in the range of between 15-20 shore on the “00” scale. This provides a material 166 that is extremely soft and compliant and intrinsically “sticky”. An extremely low shore hardness of the third central seal member 165 material 166 allows the third central seal member 165 to be easily compressed upon itself or upon an inserted instrument. For illustrative purposes only, the nature of the material 166 of the third central seal member 165 can be compared to a gelatinous substance. The material 166 exhibits a “selfclosing” nature in that it sticks occlusively to itself forming a nearly fluid/gas tight seal under very light compression.

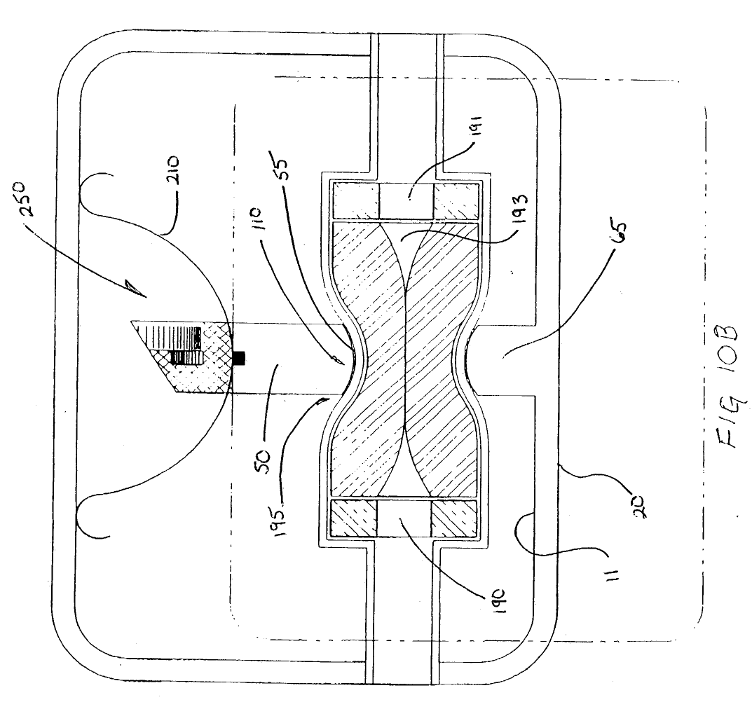

With particular reference to FIGS. 10A, 10B, 16-19, the highly compliant third central seal member 165 seals around a variety of profile shapes 192 and diameters 194 of the lumen 193 when at least one side of compressive force 67 is exerted upon the central region 195 with respect to the central portion 110 of the containment structure 160. The compressive load may be supplied by a movable, sliding or hinged, actuator 50 that maintains a compressive load upon the third central seal member 165 under the influence of a spring 210 or other resilient material. The spring 210 provides a compressive load between the actuating member 55 of the actuator 50 and the stationary member 65 positioned in the interior wall 11 of the housing 20.

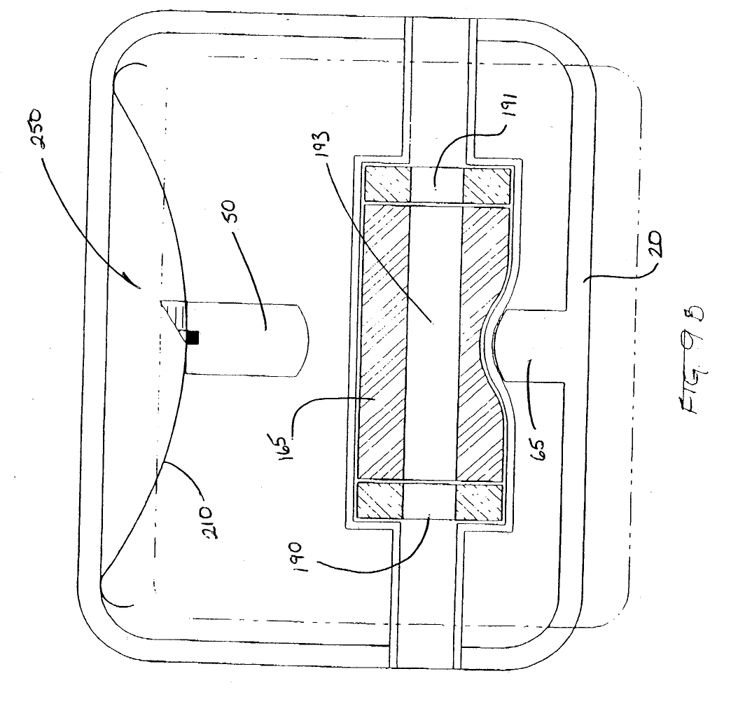

With reference to FIGS. 9A, 9B, 10A, 10B, 11, the compressive load upon the third central seal member 165 is, in one option, selectively relieved by moving the movable actuator 50 so as to compress the spring 210 and subsequently enlarge the distance between the actuating member 55 of the actuator 50 and the stationary member 65 positioned in the interior wall 11 of the housing 20. A “hold-open” or “hold-closed” feature is, in one option, a latching or detent arrangement 250. An operator can choose to have the lumen of the composite seal remain substantially open, allowing gas or fluid flow in either direction. The operator can subsequently “squeeze” or otherwise operate the actuator 50 to the following sequential position of the detent arrangement 250 thereby allowing the spring 210 to fully compress the third central seal member 165. For example, the action of “hold and release” is repeated as the actuator 50 is urged from one extreme position to another extreme position within the detent arrangement 250. The detent arrangement 250 includes, but is not limited to, a series of ramps and slides that move the sliding actuator 50 through a path.

In another embodiment, the actuator 50 includes an extension configured to be urged up an incline ramp 251 and into a depression 252 where it finds a neutral resting place under the return force of the compression spring 210. Upon further urging forward, the extension 52 of the actuator 50 is forced against an angular wall 253 that forces the extension of the actuator 50 to one side, over a ledge 255, and into a return incline ramp 256. The neutral bias of the actuator 50 is to position the extension so as to move up the first incline ramp 251 upon subsequent or further actuation of the actuator 50.

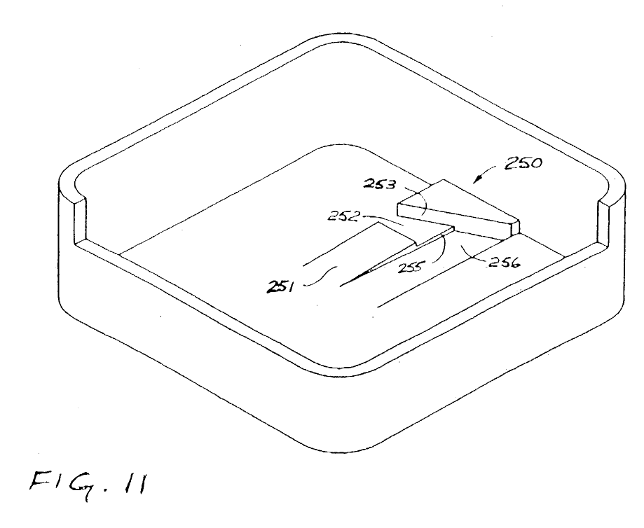

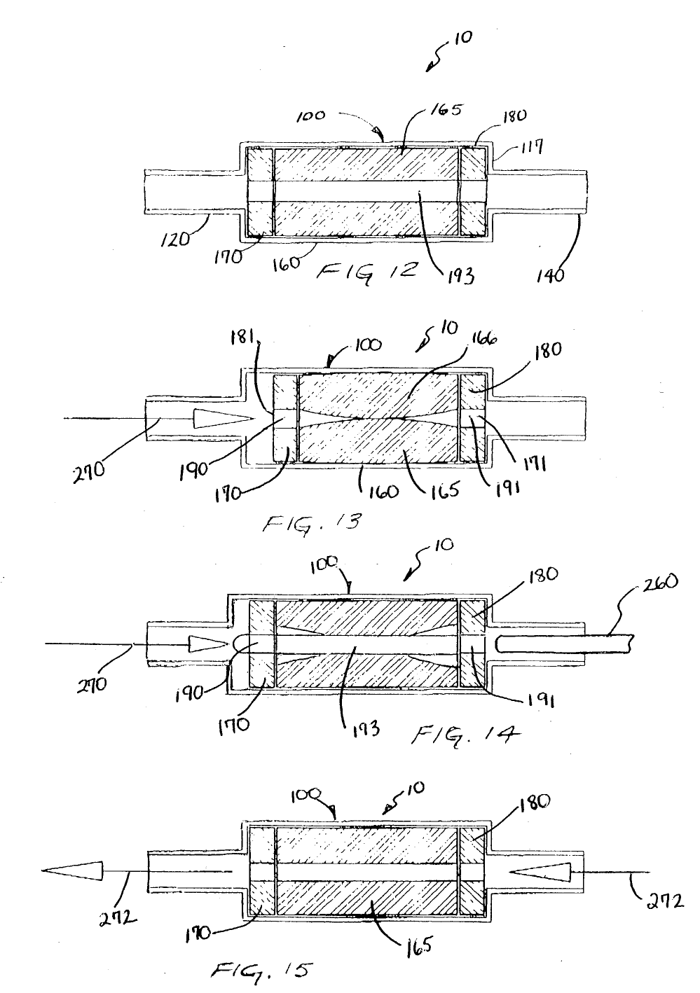

FIGS. 12-15 illustrate the use of a seal module 100 that requires no compressive load for use in sealing the stasis valve 10 closed. The non-compressive embodiment may include a second seal member 180 in a fixed position within the containment structure 160 toward the distal end 117 of the containment structure 160, a first seal member 170 in a sliding relationship within the containment structure 160, and a third central seal member 165 comprised of a highly deformable material 166. The first seal member 170 includes an elastomeric seal that is movable within the containment structure 160 in response to a retrograde flow 270. The first seal member 170 includes a length that maintains axial alignment within the containment structure 160 which, in one option, includes an orifice 181 that is significantly smaller than the lumen 193, 191 size of the other seal members 165, 180. The region adjacent to the small orifice 181 includes a thin cross-section to reduce entry force, friction and restriction. In another option, the seal module 100 includes a first seal member 170 with a first diameter, a second seal member 180 with a second diameter, and a third seal member 165 with a third diameter, the third diameter of the third seal member 165 being greater than at least one of the first diameter and the second diameter.

The seal module 100 includes, in one option, the first seal member 170 having a first material, the second seal member 180 having a second material, and the third seal member 165 having a third material, wherein at least one of the first material of the first seal member 170 and the second material of the second seal member 180 is different than the third material of the third seal member 165. The first material of the first seal member 170 and the second material of the second seal member 180 can have a lower friction that the third material of the third seal member 165. The second seal member 180 includes an elastomeric seal that is fixed within the containment structure 160 so that it does not move within the seal module 100. The second seal member 180 has a length that keeps it axially stable within the containment structure 160 and an orifice 171 that represents the designated lumen 191 size of the instrument 260.

The back pressure from the retrograde flow 270 forces the first seal member 170 toward the third central seal member 165 in the containment structure 160. As the first seal member 170 moves distally, or toward the second seal member 180 under the influence of the pressure from the gas or fluid, the third central seal member 165 is compressed. However, since the material 166 of the third central seal member 165 is essentially non-compressible, the lumen 193 of the third central seal member 165 collapses upon itself circumferencially. The material 166 of the third central seal member 165 is sufficiently soft and compliant to deform under the movement of the first seal member 170. In one option, at least one of the first and the second materials have a higher durometer than the third material. As long as there is backpressure against the first seal member 170, a gas or fluid tight seal is maintained.

In one embodiment, instrument 260, for example, a catheter or guidewire is inserted into the valve antegrade, for example, in the distal end 117 of the containment structure 160 while the lumen 193, 190, and 191 of the seal members 165, 170, and 180, are in an open configuration. The instrument 260 frictionally engages the lumen 190 of the first seal member 170 and forces it distally away from the second seal member 180 and the third central seal member 165. The first seal member 170 forms a seal against the instrument 260. The back pressure from the retrograde flow 270 against the first seal member 170 forces the first seal member 170 toward the distal end 117 of the containment structure 160 compressing the third central seal member 165 and collapsing it circumferencially against the instrument 260 forming a second, complete seal.

In another embodiment, instrument 260, for example, a catheter or guidewire is inserted into the valve antegrade, for example, in the distal end 117 of the containment structure 160 while the lumen 193 of the third central seal member 165 is in a closed configuration. The instrument 260 frictionally engages the lumen 190 of the first seal member 170 and forces it distally away from the second seal member 180 and the closed third central seal member 165. The first seal member 170 forms a seal against the instrument 260. The back pressure from the retrograde flow 270 against the first seal member 170 forces the first seal member 170 toward the distal end 117 of the containment structure 160 compressing the third central seal member 165 and collapsing it circumferencially against the instrument 260 forming a second, complete seal.

In yet another embodiment, two or more instruments 260 are inserted into the valve antegrade, for example, in the distal end 117 of the containment structure 160 while the lumen 193, 190, and 191 of the seal members 165, 170, and 180, are in an open configuration, or in another option, while the lumen 193 of the third central seal member 165 in a closed configuration. The instruments 260 frictionally engage the lumen 190 of the first seal member 170 and forces it distally away from the second seal member 180 and the third central seal member 165. The first seal member 170 forms a seal against the instruments 260. The back pressure from the retrograde flow 270 against the first seal member 170 forces the first seal member 170 toward the distal end 117 of the containment structure 160 compressing the third central seal member 165 and collapsing it circumferencially against the instruments 260 forming a second, complete seal. The material 166 of the third central seal member 165 is so compliant that it forms a seal around the instruments 260 even if the instruments 260 are irregularly shaped.

An pressure from the antegrade flow 272 repositions the first seal member 170 toward the proximal end 115 of the containment structure 160 and subsequently opens the stasis valve 10 while preventing back-flow or leakage. While this arrangement may not be as friction-less as the other embodiments using an actuator 50, it may offer the advantages of “hands-free” operation.

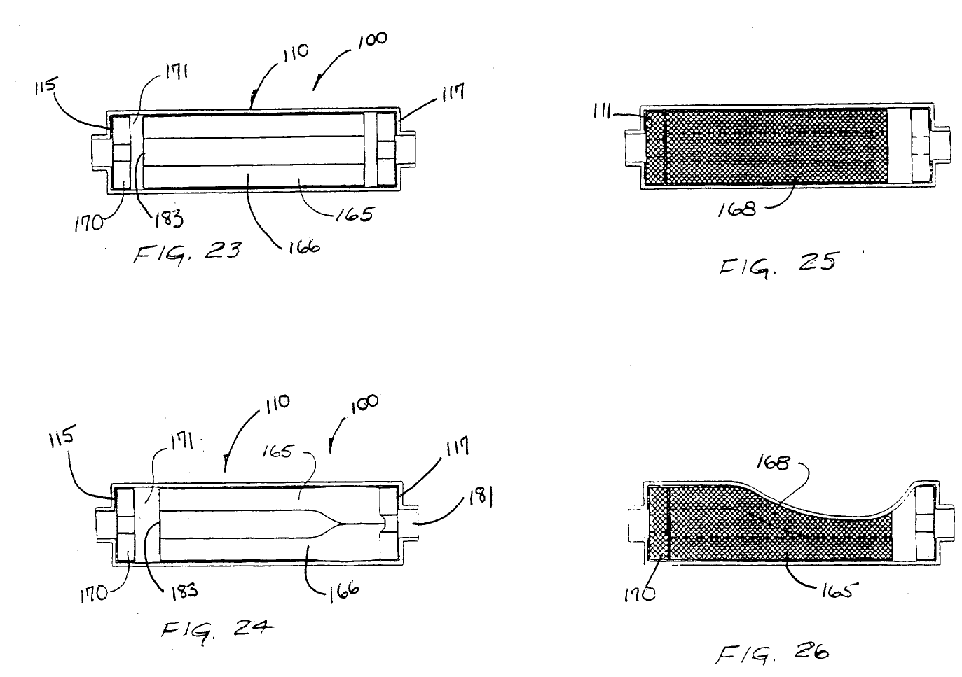

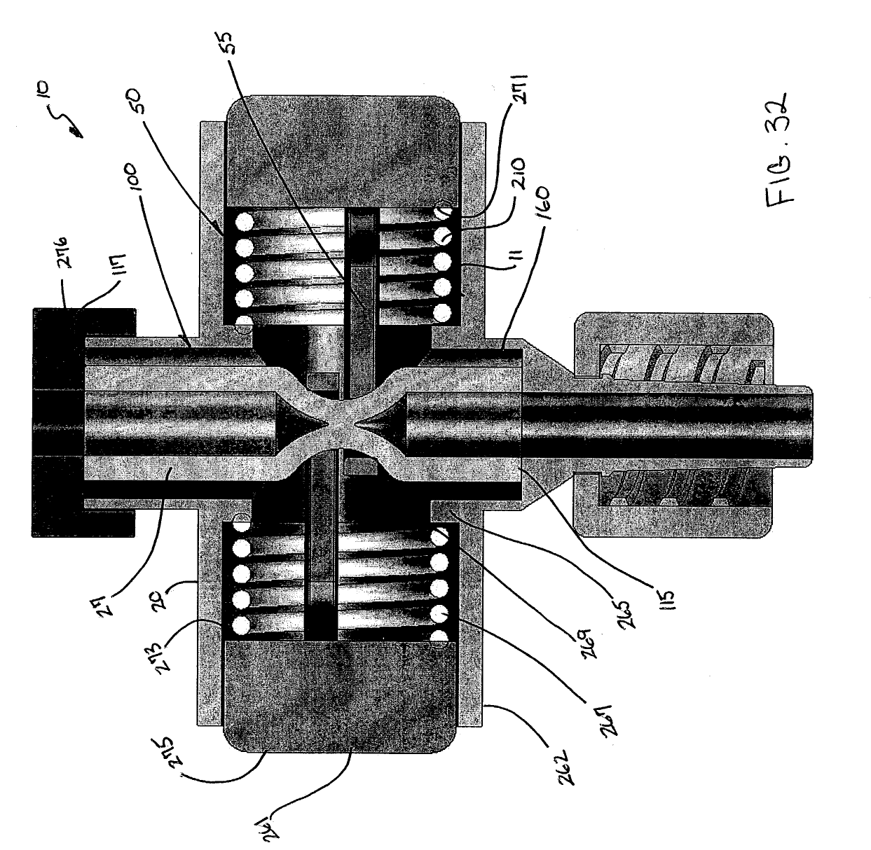

With reference to FIGS. 23-29, the stasis valve 10 includes the seal module 100 enclosed in the housing 20 where, in one option, the seal module 100 includes the first seal member 170 at the proximal end 115 of the containment structure 160, a second seal member 180 at the distal end 117 of the containment structure 160, and a third central seal member 165 extending between the first and the second seal members 170, 180. The plurality of seal members 165, 170 and 180 have an internal diameter sized to allow the passage of fluids or gases. A support member 168 includes a woven or braided material 166 configured to fit over the first seal member 170 and over the third central seal member 165. The support member 168 is capable of retractionably collapsing with a compressive side-load by opposing protrusions, for example, the actuating member 55 and the stationary member 65. In one option, the support member 168 is compressible under a side-load but not elongatable. In one example, this is accomplished by a biased weaving or tubular braiding of rigid material. An example of such a construction is the shielding found on certain electronic wire components. A tubular braided or woven rigid material exhibits the characteristics of an elasometric material and yet is not, itself, elastic.

The actuator 50 is adapted to move from a first position to a second position. In the first position the actuator is, in one option, disposed and held against a portion of the seal module 100 depressing or collapsing, for example, an off-center portion 109 of the containment structure 160 by a compressive force 67 (e.g. by a spring 210). In the second position, the actuator 50 is disposed away from a portion of the seal module by a compressive force 67 (e.g. by depressing the actuator flange 57) thus allowing, for example, the off-center portion 109 of the containment structure 160 to retract to an uncollapsed configuration.

In the first position, the actuator 50 is, in another option, disposed and held against a portion of the seal module 100 which depresses and at least partially collapses, for example, the central portion 110 of the containment structure 160 by a compressive force 67 (e.g. by a spring 210). In the second position, the actuator 50 is disposed away from a portion of the seal module 100 by a compressive force 67 (e.g. by depressing the actuator flange 57) thus allowing, for example, the central portion 110 of the containment structure 160 to retract to an uncollapsed configuration.

In one embodiment, the first seal member 170 is fixed in a position at the proximal end 115 of the central portion 110 of the seal module 100. The first abutment 111 forms a stop or seat for a first seal member 170. The braided or woven support member 168 is connected to the first seal member 170 and attached to or formed into the wall of the third central seal member 165. The third central seal member 165 is thus not permitted to migrate under a backpressure load into the distal orifice 181 of the second seal member 180 and occlude said orifice 171. In one example, a compressive side-load is applied to the third central seal member 165. In another example, a compressive side-load is applied by opposing protrusions, for example, actuator 50 and stationary member 65, under a spring 210 load. Under this influence, the material 166 of the third central seal member 165 is not allowed to extrude longitudinally to an area 182 due to the linear limit of the braided or woven support member 168.

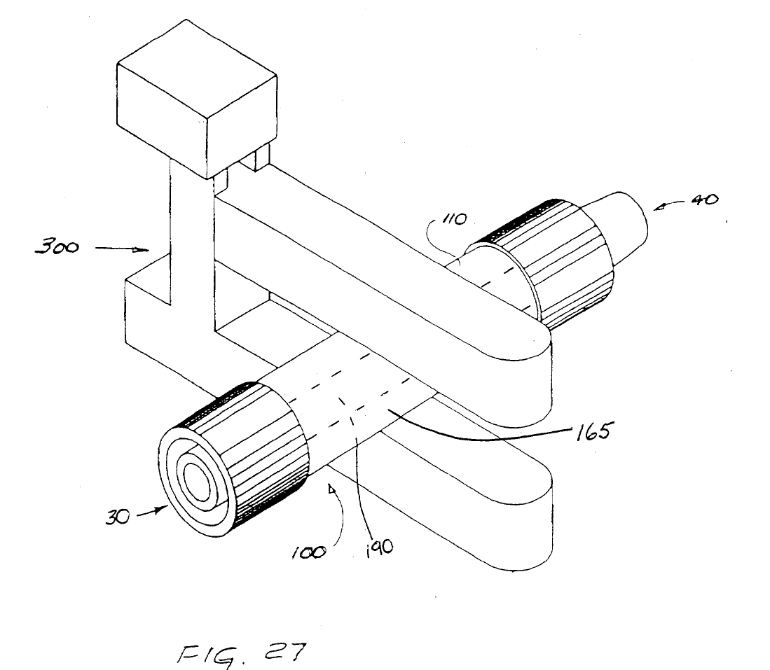

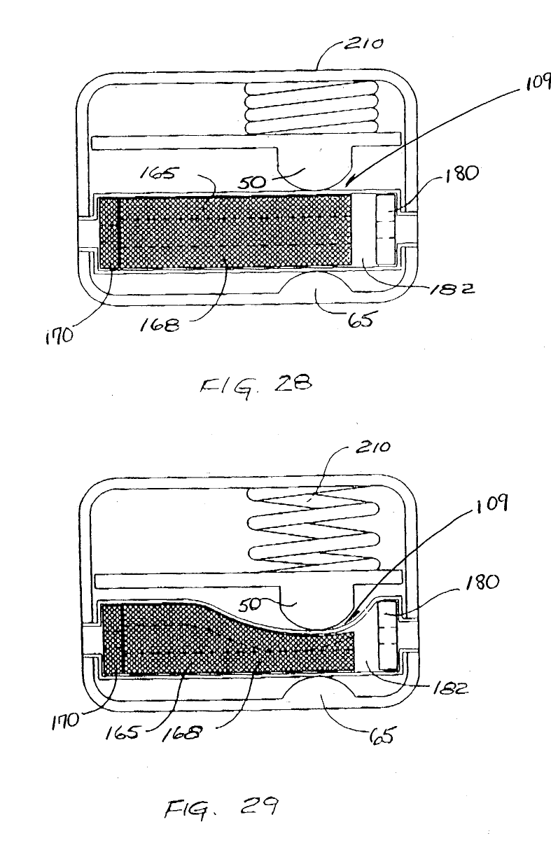

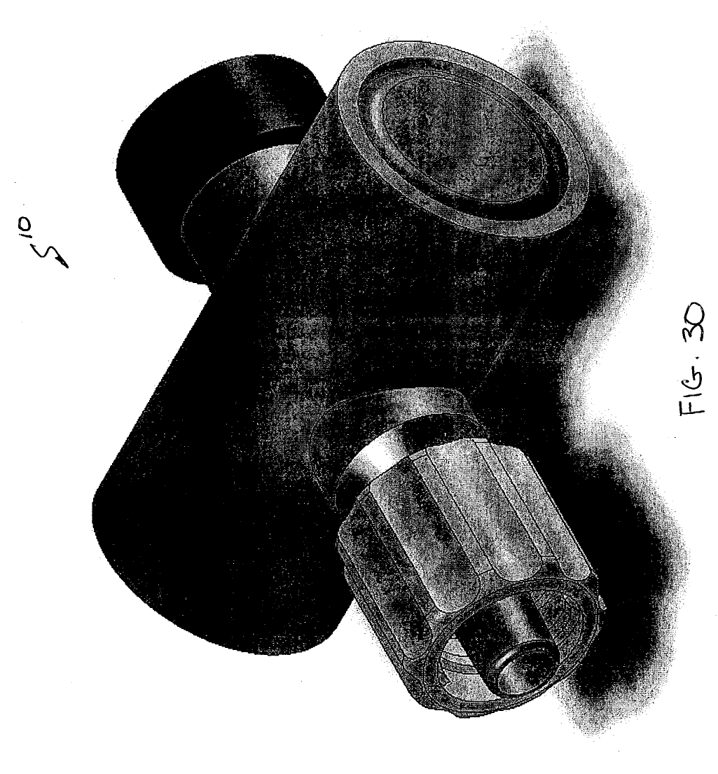

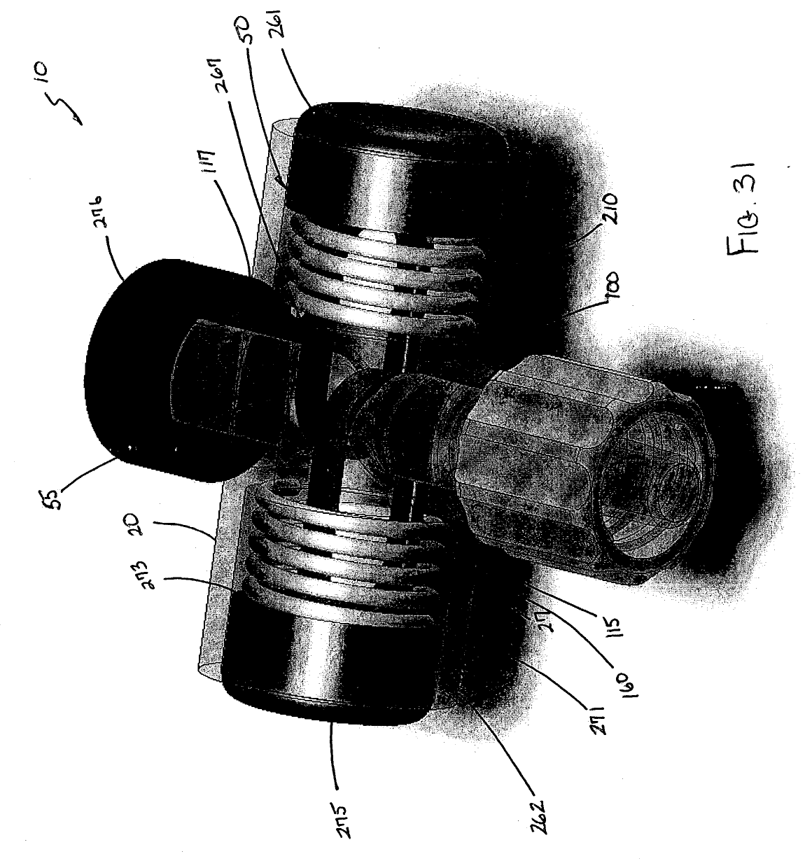

Now referring to FIG. 27, the seal module 100 is, in one option, used without the housing 20 or the containment structure 160. The seal module 100 includes an elongate tubular structure 101 having a central portion 110 with a proximal end 30 and a distal end 40. In one option, a compressive or occlusive side load or “squeezing” is supplied by a separate tool or device, such as a clamp 300, forceps, hemostat or a combination thereof, or additionally occluded by bending or finger pressure. The third central seal 165 is, in one option, closed off from the central lumen 193 of the seal module 100 in the instance where a plurality of instruments 260 are within said lumen 193. The highly occlusive nature of the material 166 of the third central seal member 165 allows it to conform to the interstices adjacent to the instruments. For instance, a guidewire and catheter may be placed into the same lumen 193 for extension into a body passage rather than have two or more separate insertion sites into the same vessel or passage.

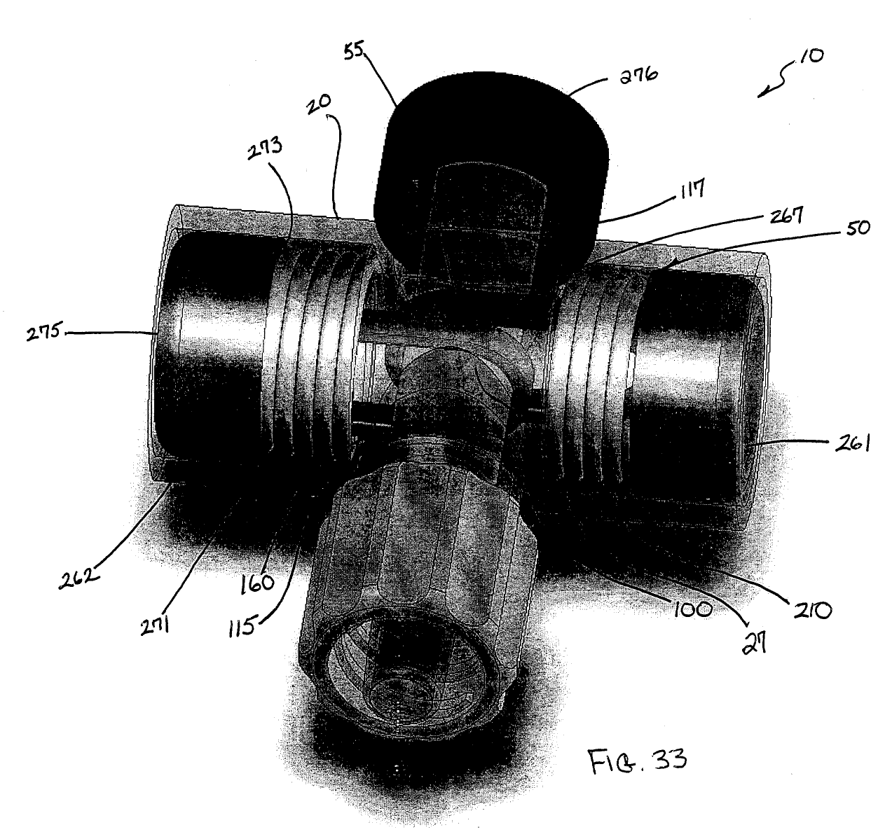

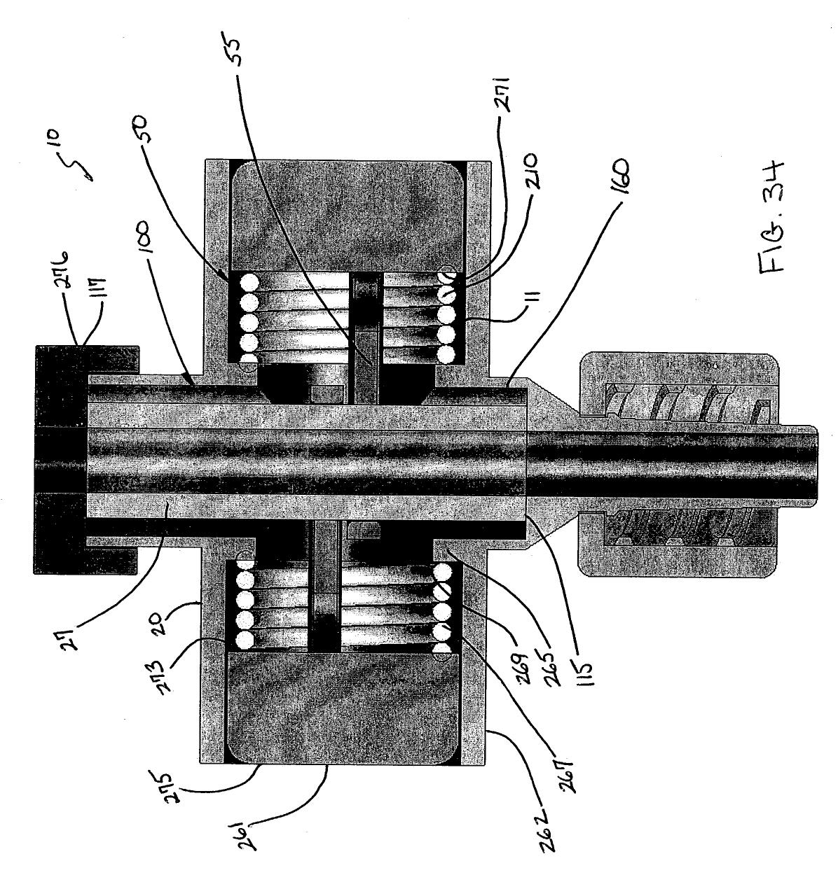

FIGS. 30-34 illustrate one embodiment of the stasis valve 10 including a seal module 100 having a lumen sized to allow the passage of fluids or gases. The seal module 100 includes a containment structure 160 with a proximal end 115 and a distal end 117. The seal module 100 is formed of one or more seal members, as discussed above. In another option, the seal module 100 and/or any of its respective seal members can be formed of one or more materials, including their relative properties, as discussed above.

In one option, two circular actuators 50 are at least partially circumferencially disposed about a portion of the seal module 100 movable from a first position to a second position on opposing sides of the housing 20. The actuators 50 each include an actuating member 55 which, in one option, is U-shaped. The outer wall 262 of the housing 20 and the inner flange wall 265 of the housing 20 provides opposing support for two resilient members 267 (e.g. spring 210) disposed within the actuating member 55. The resilient members 267 include a proximal end 269 and a distal end 271 where the proximal end 269 of the resilient members 267 abut the inner flange wall 265 of the housing 20 and the distal end 271 of the resilient members 267 each abut the proximal end 273 of an actuator button 261. In one option, the actuators 50 are configured cylindrically to slide along the cylindrical interior wall 11 of the housing 20 from a first position to a second position.

In the first position the actuating members 55 of the actuators 50 are, in one option, disposed and at least partially circumferencially disposed about the portion 108 of the seal module 100 depressing and at least partially collapsing a portion 108 of the containment structure 160 by a compressive force 67 (e.g. by a spring 210). The lumen 193 of the third seal member 165 is at least partially collapsed by the compressive force 67. In the second position, the actuators 50 are disposed away from the portion 108 of the seal module 100 by a compressive force 67 (e.g. by depressing the distal end 275 of the actuator button 261). As each actuator button 261 is depressed, each actuator 50 slides along the cylindrical interior wall 11 of the housing 20. The proximal end 273 of each actuator button 261 compresses the distal end 271 of each resilient member 267 which in turn, the proximal end 269 of each resilient member 267 compresses against the inner flange wall 265 of the housing 20. Such movement allows each engaged actuating member 55 to forcibly disengage opposing outer walls 27 of the seal module 100 allowing the portion 108 of the containment structure 160 to retract to an uncollapsed configuration where gases and fluids can pass therethrough. As the actuator 50 is disposed away from the portion 108 of the seal module 100, the lumen 193 of the third seal member 165 is able to retract in an unsealed configuration.

In another embodiment, the stasis valve 10 includes a containment structure 160 with a proximal end 115 and a distal end 117 with only one actuators 50 disposed against a portion of the seal module 100 movable from a first position to a second position. The actuator 50 includes an actuating member 55 which, in one option, is U-shaped. The outer wall 262 of the housing 20 and the inner flange wall 265 of the housing 20 provide an opposing support for the resilient member 267 (e.g. spring 210) disposed within the actuating member 55. The resilient member 267 includes a proximal end 269 and a distal end 271 where the proximal end 269 of the resilient member 267 abuts the inner flange wall 265 of the housing 20 and the distal end 271 of the resilient member 267 is disposed against the proximal end 273 of an actuator button 261.

In one option, the actuator 50 is configured cylindrically to slide along the cylindrical interior wall 11 of the housing 20 from a first position to a second position. In the first position the actuating member 55 of the actuator 50 is, in one option, disposed and held against the portion 108 of the seal module 100 depressing and at least partially collapsing a portion 108 of the containment structure 160 by a compressive force 67 (e.g. by a spring 210). In the second position, the actuator 50 is disposed away from the portion 108 of the seal module 100 by a compressive force 67 (e.g. by depressing the distal end 275 of the actuator button 261).

As the actuator button 261 is depressed, the actuator 50 slides along the cylindrical interior wall 11 of the housing 20. The proximal end 273 of the actuator button 261 compresses the distal end 271 of the resilient member 267 which in turn, the proximal end 269 of the resilient member 267 compresses against the inner flange wall 265 of the housing 20. Such movement allows the engaged actuating member 55 to forcibly disengage the outer wall 27 of the seal module 100 allowing the portion 108 of the containment structure 160 to retract to an uncollapsed configuration where gases and fluids can pass therethrough.

The actuating member 55 and/or the actuating button 261 in one option includes aluminum. In another option, the actuating member 55 and the actuating button 261 include plastic. The housing 20, in one option, is made of ABS plastic. In one option, the third central seal member 165 includes material 166 that is highly elastic, deformable, compliant and yet virtually non-compressible. Materials 166 include modified vinyl, silicone, polyurethane or a combination thereof. The basic materials are, in one option, modified by compounding them with waxes and/or oils or un-cross-linked modifiers. Such materials are commonly available as “C-Flex” or “Kraton” in the range of 5 to 15 (shore A), as examples. The shore hardness of the material 166 is, in another option, in the range of between 15-20 shore on the “00” scale.

The stasis valve 10, in one option, is made from machining pre-existing amounts of metals and/or plastics. For example, The actuating member 55 and the actuating button 261 is machined from aluminum. In another example, the actuating member 55 and the actuating button 261 are machined from plastic where the housing 20, in one option, is machined from ABS plastic. In another example, the housing 20, actuator button 261, the connecting member 35 and a cap 276 are injection molded utilizing the various material outlined above.

In an example where the stasis valve 10 includes two actuators 50, the stasis valve 10 is assembled by inserting the actuator button 261 and resilient member 267 (e.g., spring 210) into one side of the housing 20. The actuator button 261 and resilient member 267 (e.g., spring 210) are inserted into an opposing side of the housing 20. Each actuator button 261 is completely compressed and held while the seal module 100 is inserted through the housing 20 and between each actuator 50. Each actuator button is released and the cap 276 secured to the housing 20, for example, with an adhesive. Further, the connecting member 35 is snapped onto the housing. The materials used and the assembly thereof of the stasis valve 10 as described herein can include any of the earlier disclosed embodiments or a combination thereof.

It is to be understood that the above description is intended to be illustrative, and not restrictive. Many other embodiments will be apparent to those of skill in the art upon reading and understanding the above description. It should be noted that embodiments or portions thereof discussed in different portions of the description or referred to in different drawings can be combined to form additional embodiments of the invention. The scope of the invention should, therefore, be determined with reference to the appended claims, along with the full scope of equivalents to which such claims are entitled.

We claim:

1. A valve comprising:
a seal module having a lumen sized to allow the passage of fluids or gases, the seal module having a proximal end and a distal end;
a resilient member disposed against an actuator; and
the actuator disposed against a portion of the seal module, the actuator movable from a first position to a second position, in the first position the actuator is disposed against the portion of the seal module collapsing the lumen between the proximal end and the distal end of the seal module creating a seal, in the second position the actuator is disposed away from the portion of the seal module by a force allowing the lumen to return to an unsealed configuration.

2. The valve as recited in claim 1, wherein the actuator is disposed away from the portion of the seal module by a compressive force.

3. The valve as recited in claim 1, wherein the seal module is enclosed in a housing.

4. The valve as recited in claim 1, wherein the resilient member is a spring.

5. The valve as recited in claim 1, further comprising at least one detent, the detent retaining a lumen of the seal module in at least one of a closed position or an open position.

6. The valve as recited in claim 1, wherein the actuator includes an extension configured to slidably engage into a first incline ramp extending from an interior wall of a housing and an angular wall with a depression having a ledge extending therebetween, where the extension of the actuator is further configured to engage over the ledge of the depression and into a second incline ramp.

7. The valve as recited in claim 1, wherein the seal module is collapsible by the actuator and by an opposing protrusion.

8. The valve as recited in claim 1, wherein the actuator includes an actuating member having a U-shaped configuration.

9. The valve as recited in claim 1, having two actuators disposed on opposing sides of a housing.

10. A valve comprising:
a seal module having a plurality of seal members including a first seal member, a second seal member, and a third seal member extending between the first and the second seal members, the plurality of seal members each having a lumen sized to allow the passage of fluids or gases; and
the first seal member having a first material, the second seal member having a second material, and the third seal member having a third material, wherein at least one of the first material of the first seal member and the second material of the second seal member is different than the third material of the third seal member.

11. The valve as recited in claim 10, wherein the first seal member is disposed in a sliding relationship with a proximal end of the seal module, the second seal member is disposed against a distal end of the seal module, and the third seal member extends between the first and the second seal members, the third seal member collapsible under pressure from the first seal member.

12. The seal module as recited in claim 10, wherein at least one of the first and the second materials have a higher durometer than the third material.

13. The seal module as recited in claim 10, wherein the first seal member has a first diameter, the second seal member has a second diameter, and the third seal member has a third diameter, the third diameter of the third seal member is greater than at least one of the first diameter and the second diameter.

14. The seal module as recited in claim 10, wherein at least one of the first material of the first seal member and the second material of the second seal member has a lower friction than the third material of the third seal member.

15. The valve as recited in claim 10, further comprising a support member fitted over the first and the third seal members.

16. The valve as recited in claim 15, wherein the support member includes a woven material, a braided material, or a combination thereof.

17. A valve comprising:
a seal module having a lumen sized to allow the passage of fluids or gases, the seal module having a proximal end and a distal end;
one or more resilient members disposed against one or more actuators; and
the one or more actuators disposed against a portion of the seal module, the one or more actuators movable from a first position to a second position, in the first position the one or more actuators are at least partially circumferencially disposed about the portion of the seal module collapsing the lumen between the proximal end and the distal end of the seal module creating a seal, in the second position the one or more actuators are circumferencially disposed away from the portion of the seal module allowing the lumen to return to an unsealed configuration.

18. The valve as recited in claim 17, wherein the one or more actuators each include an actuating member having a U-shaped configuration.

19. The valve as recited in claim 17, wherein the one or more resilient members is a spring.

20. The valve as recited in claim 17, wherein the one or more actuators are disposed on opposing sides of a housing.

21. The valve as recited in claim 17, wherein each of the one or more actuators is disposed away from the portion of the seal module by a compressive force.

22. A method comprising:
providing a seal module having a plurality of seal members including a first seal member with a first lumen in a sliding relationship with a proximal end of the seal module;
disposing a second seal member with a second lumen against a distal end of the seal module;
disposing a third deformable seal member with a third lumen between the first and the second seal members, the plurality of seal members sized to allow the passage of fluids or gases;
urging the first seal member toward the second seal member where the deformable third seal member is compressed; and
collapsing the third seal member creating a seal of at least a portion of the third lumen of the third seal member.

23. The method as recited in claim 22, wherein sealing the module includes sliding the first seal member disposed against a proximal end of the seal module toward the second seal member disposed against the distal end of the seal module and collapsing the third seal member extending between the first and the second seal members.

24. The method as recited in claim 22, further comprising disposing an instrument through a first diameter of the first seal member, a second diameter of the second seal member, and a third diameter of the third seal member sealing at least the first seal member to the instrument.

25. The method as recited in claim 22, including moving an actuator along an incline ramp of an interior wall of a housing and into a depression, disposing the actuator under the force of a compression spring, urging the actuator against an angular wall, pushing the actuator to one side over a ledge, and disposing the actuator into a return incline ramp.

26. A method comprising:
forming a seal module having a lumen sized to allow the passage of fluids or gases;
disposing an actuator against a portion of the seal module with a resilient material, the actuator movable from a first position to a second position;
collapsing the lumen between a proximal end and a distal end of the seal module creating a seal; and
moving the actuator away from the portion of the seal module in the second position allowing the lumen to return to an unsealed configuration.

27. The method as recited in claim 26, wherein moving the actuator away from the portion of the seal module includes moving the actuator by a compressive force.

28. The method as recited in claim 26, wherein collapsing the seal module includes depressing the seal module with the actuator and an opposing protrusion.

29. The method as recited in claim 26, wherein collapsing the seal module includes depressing the seal module with two opposing actuators.

30. The method as recited in claim 26, further comprising moving at least one detent, and retaining the lumen of the seal module in at least one of a closed position or an open position.

31. The method as recited in claim 26, including moving an extension of the actuator along an incline ramp of an interior wall of a housing and into a depression, disposing the extension under the force of a compression spring, urging the extension against an angular wall, pushing the extension to one side over a ledge, and disposing the extension into a return incline ramp.
